(19) United States
(12) Patent Application Publication
(10) Pub. No.: US 2026/0124130 A1
(43) Pub. Date: May 7, 2026

(54) COMPOSITION COMPRISING A NON-IONIC SURFACTANT, A HYDROCARBON-BASED OIL, A BUTTER AND A WAX

(71) Applicants: Nathalie SCHEIDLER-FOEGLE, Chevilly la Rue (FR); Maria Helena ROCHA BASTIEN, Chevilly la Rue (FR); L’OREAL, Paris (FR)

(72) Inventors: Nathalie SCHEIDLER-FOEGLE, Chevilly la Rue (FR); Maria Helena ROCHA BASTIEN, Chevilly la Rue (FR)

(21) Appl. No.: 19/118,392

(22) PCT Filed: Oct. 13, 2023
(86) PCT No.: PCT/EP2023/078453

§ 371 Date: Apr. 4, 2025

(30) Foreign Application Priority Data
Oct. 13, 2022 (FR) FR2210549

(51) Int. Cl. A61K 8/92; A61K 8/06; A61K 8/34; A61K 8/37; A61K 8/60; A61K 8/85; A61Q 19/08

(52) U.S. Cl. CPC A61K 8/922; A61K 8/062; A61K 8/342; A61K 8/375; A61K 8/604; A61K 8/85; A61K 8/927; A61Q 19/08; A61K 2800/5922; A61K 2800/596

(57) ABSTRACT

The present invention relates to a composition, preferably a cosmetic composition, in particular an anti-aging composition, comprising at least one non-ionic surfactant, at least one non-volatile hydrocarbon-based oil, at least one cupuaçu butter and at least one wax. It also relates to an associated method and use.

The present invention relates to a composition, preferably a cosmetic composition, in particular an anti-aging composition, comprising at least one non-ionic surfactant, at least one non-volatile hydrocarbon-based oil, at least one cupuaçu butter and at least one wax.

The human skin is composed of two compartments, namely a surface compartment (the epidermis) and a deep compartment (the dermis).

The natural human epidermis is primarily composed of three types of cells which are keratinocytes, which are largely predominant, melanocytes, and Langerhans cells. Each of these cell types contributes via the specific functions thereof to the key role played by the skin in the body. The dermis also provides a solid support for the epidermis. It also supplies nourishment for it. It is composed mainly of fibroblasts and an extracellular matrix, itself composed mainly of collagen, elastin and a substance, known as fundamental substance, components formed by the fibroblast.

Collagen fibers ensure the strength of the dermis, but also the elasticity and tonicity of the skin and/or mucosa.

Collagen fibers are constantly renewed but this renewal declines with age which causes thinning of the dermis. Furthermore, various factors cause collagen degradation, with all the effects that may be envisaged on the structure and/or firmness of the skin and/or mucosa. During the aging process, various characteristic signs appear on the skin, expressed in particular by a modification of the structure and functions of the skin. The main clinical signs of skin aging are particularly the appearance of lines and/or wrinkles, increasing with age. These wrinkles and lines can be deep, moderate or superficial, and are expressed by a depression or creases on the skin surface. Furthermore, a loss of volume of the upper part of the face is observed, particularly at the cheekbones and cheeks.

Various solutions are used to combat skin aging. Of these, a variety of cosmetic anti-aging compositions are found. Some of these formulas comprise a high oil and wax content, and often have dosage forms with overly firm finger uptakes—therefore sometimes difficult to extract—as well as a greasy and heavy texture, penetrating slowly, i.e., they remain on the surface of the skin which causes a loss of time for the consumer's beauty routine, or penetrating too quickly, i.e., the consumer does not have enough time to spread the film on the skin, thus causing a non-homogeneous application on the skin. Furthermore, the film of these compositions is perceived as greasy, shiny, thick and remanent.

To remedy this drawback, powders (i.e., fillers) are often introduced into the compositions. However, the combination of a film on the skin and fillers can generate fluff during application. This can make the subsequent application of makeup in the beauty routine difficult.

There is therefore a need for compositions having a texture that is soft and of easy uptake, nourishing and covering, while having an optimized penetration, and which do not fluff during application. In particular, there is a need for compositions which form a covering film on the skin which is light, non-greasy and having a matt appearance, even without the use of fillers.

The inventors have now discovered that introducing a cupuaçu butter and at least one wax, such as beeswax, into oil-in-water type emulsions, gives the compositions a nourishing and covering texture, while having an optimized penetration. On the skin, the film obtained is light and non-greasy, and does not fluff. The skin has a matter appearance, even without the use of fillers. Finally, the compositions are easy to extract and to apply.

Thus, the present invention relates to a composition in the form of an oil-in-water emulsion comprising:
at least one non-ionic surfactant,
at least one non-volatile hydrocarbon-based oil,
at least one cupuaçu butter, and
at least one wax.

The composition according to the invention is preferably cosmetic. Preferably, the composition is an anti-aging composition.

The term “oil-in-water emulsion” denotes a composition comprising an oily phase dispersed in a continuous aqueous phase.

The present invention also relates to a cosmetic method for cleansing keratin materials, preferably the skin, comprising the application of a composition according to the invention on said keratin materials.

The present invention also relates to the cosmetic use of a composition according to the invention as an anti-aging composition.

### Non-Ionic Surfactant

The composition according to the invention comprises at least one non-ionic surfactant. Preferably, it comprises at least two non-ionic surfactants.

Preferably, the non-ionic surfactant is selected from fatty acid and polyethylene glycol esters, alkylC8-C30(poly)glycosides and mixtures thereof.

The non-ionic surfactant can be selected from fatty acid and polyethylene glycol esters. In this case, preferably, it also comprises an additional surfactant selected from C₁₆-C₂₂ fatty acid and glyceryl esters.

The fatty acid and polyethylene glycol ester is preferably a C₁₆-C₂₂ fatty acid ester including 8 to 200 ethylene oxide units. The fatty chain of esters can be selected particularly from stearyl, behenyl, arachidyl, palmityl, cetyl structural units and mixtures thereof, such as cetearyl, and preferably a stearyl chain.

The number of ethylene oxide units can vary from 8 to 200, preferably from 10 to 150, and even better from 10 to 120. According to one particular embodiment of the invention, this number can vary from 20 to 110.

As an example of a fatty acid and polyethylene glycol ester, mention may be made of stearic acid esters comprising 20, 30, 40, 50 or 100 units of ethylene oxide respectively, such as products marketed under the trade name Myrj 49 P (polyethylene glycol stearate 20 OE; CTFA name: PEG-20 stearate), Myrj 51, Myrj 52 P (polyethyleneglycol stearate 40 OE; CTFA name: PEG-40 stearate), Myrj 53 or Myrj 59 P by CRODA.

The fatty acid and polyethylene glycol ester may be present in the composition according to the invention in a content ranging from 0.1% to 10% by weight, with respect to the total weight of the composition, and preferably ranging from 0.5% to 8% by weight, and preferably ranging from 0.5% to 5% by weight, and preferably ranging from 0.8% to 3% by weight.

Preferably, the composition according to the invention also comprises an additional emulsifier selected from C₁₆-C₂₂ fatty acid and glyceryl esters.

According to a preferred embodiment, the composition comprises a glyceryl and fatty acid ester, which can be obtained particularly using an acid including a saturated linear alkyl chain, with 16 to 22 carbon atoms. As a glyceryl and fatty acid ester, particular mention may be made of glyceryl stearate (glyceryl mono-, di- and/or tri-stearate) (CTFA name: Glyceryl stearate), glyceryl ricinoleate, and mixtures thereof. Preferably, the glyceryl and fatty acid ester used is selected from glyceryl stearates.

The glyceryl and fatty acid ester can be present in a quantity ranging from 0.1 to 10% by weight, with respect to the total weight of the composition, preferably ranging from 0.1 to 5% by weight, and preferably ranging from 0.5% to 3% by weight.

In particular, the composition according to the invention may comprise a mixture of glyceryl stearate and polyethylene glycol monostearate 100 OE, and in particular that comprising a 50/50 mixture, marketed under the trade name Arlacel 165 by Croda.

The non-ionic surfactant may also be selected from alkylC8-C30(poly)glycosides.

These surfactants can be more particularly represented by the following general formula:

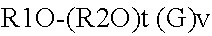

wherein R1 is a linear or branched alkyl and/or alkenyl radical including about 8 to 30 carbon atoms, an alkylphenyl radical of which the linear or branched alkyl radical includes from 8 to 24 carbon atoms; R2 is an alkylene radical including about from 2 to 4 carbon atoms; G is a sugar unit including from 5 to 6 carbon atoms; t denotes a value ranging from 0 to 10, preferably 0 to 4, preferably 0 to 3; and v denotes a value ranging from 1 to 15, preferably from 1 to 4.

Note furthermore that each unit of the polyoside portion of the alkylpolyglycoside can be of isomeric α or β form, in L or D form, and the configuration of the saccharide residue can be of the furanoside or pyranoside type.

It is of course possible to use mixtures of alkylpolyosides, able to differ from one another by the nature of the alkyl unit carried and/or the nature of the polyoside carrier chain.

According to a particular embodiment, the alkyl(poly)glycoside surfactants are compounds having the formula described hereinabove wherein R1 denotes more particularly a saturated or unsaturated, linear or branched alkyl radical including from 12 to 24 carbon atoms, t denotes a value ranging from 0 to 3 and more particularly equal to 0, G can denote glucose, fructose, galactose, maltose, maltotriose, lactose, cellobiose, mannose, ribose, dextran, talose, allose, xylose, levoglucan, cellulose or starch, preferably glucose. The degree of polymerization, i.e., the value of v in the formula hereinabove, can range from 1 to 5, preferably from 1 to 4. The average degree of polymerization is more particularly between 1 and 2.5, preferably from 1.05 to 2.5, and more preferably from 1.1 to 2.

The glycosidic bonds between the sugar units are of the 1-6 or 1-4 and preferably 1-4 type.

Use can in particular be made of coco(poly)glucoside (for example MONTANOV 82® and MONTANOV S®), arachidyl(poly)glucoside (for example MONTANOV 202®), Myristyl(poly)glucoside (for example MONTANOV 14®), cetylstearyl(poly)glucoside (or cetearyl(poly)glucoside) (for example MONTANOV 68®), C12-C20 alkyl(poly)glucosides (for example MONTANOV L®), isostearyl(poly)glucoside (for example Montanov WO 18®) or octyldodecyl(poly)xyloside (for example FLUIDANOV 20X®.

Preference is given to cetylstearyl(poly)glucoside such as the commercial product MONTANOV 68®.

Preferably, the content in alkyl(poly)glycosides varies from 0.05 to 10% by weight, preferably from 0.1 to 5% by weight, and more preferably from 0.2 to 5%, better from 0.25 to 3% by weight with respect to the total weight of the composition.

Preferably, the composition according to the invention comprises a mixture of surfactants selected from a fatty acid and polyethylene glycol ester, a glyceryl and C₁₆-C₂₂ fatty acid ester and an alkylC8-C30(poly)glycoside.

More preferably, the composition according to the invention comprises a mixture of at least one fatty acid and polyethylene glycol ester, at least one glyceryl and C₁₆-C₂₂ fatty acid ester and at least one alkylC8-C30(poly)glycoside.

Preferably, the total non-ionic surfactant content varies from 1 to 20% by weight, preferably from 2 to 15% by weight, and more preferably from 4 to 10% by weight with respect to the total weight of the composition.

### Non-Volatile Hydrocarbon-Based Oil

The composition according to the invention comprises at least one non-volatile hydrocarbon-based oil.

“Oil” refers to a non-aqueous compound, liquid at 25° C. at atmospheric pressure (1.013×10⁵ Pa), not water-miscible.

“Not miscible” means that the mixture of the same quantity of water and oil, after stirring, does not lead to a stable solution that comprises only a single phase, in the aforementioned conditions of temperature and pressure. The observation is made with the naked eye or using a phase contrast microscope if necessary, on 100 g of mixture obtained after Rayneri stirring sufficient to cause a vortex to appear within the mixture (for the purposes of information 200 to 1000 rpm); the resulting mixture being left to sit, in a closed bottle, for 24 hours at ambient temperature before observation.

“Non-volatile oil” refers to an oil of which the vapor pressure at 25° C. and atmospheric pressure, is not zero and is less than 10-3 mm of Hg (0.13 Pa).

“Hydrocarbon-based oil” refers to an oil essentially formed, or consisting, of carbon and hydrogen atoms, and optionally oxygen, nitrogen atoms, and containing no silicon or fluorine. The hydrocarbon-based oil is therefore distinct from a silicone oil and from a fluorine oil.

It may contain alcohol, ester, ether, carboxylic acid, amine and/or amide groups. Preferably, the hydrocarbon-based oil is free of heteroatoms such as nitrogen, sulfur and phosphorus.

In particular, this non-volatile hydrocarbon-based oil can comprise at least one alcohol function (it is then an “alcohol oil”) or at least one ester function (it is then an “ester oil”).

Preferably, the non-volatile hydrocarbon-based oil according to the invention is selected from:
C₁₀- C₂₆ alcohols;
ester oils having between 17 and 40 carbon atoms, preferably selected from:
   monoesters comprising between 17 and 40 carbon atoms in total, in particular monoesters with formula R₁COOR₂ wherein R₁ is the residue of a linear or branched or aromatic fatty acid including from 4 to 40 carbon atoms, optionally saturated, and R₂ is a hydrocarbon chain in particular branched containing from 3 to 40 carbon atoms, with the condition that R₁+R₂ is greater than or equal to 17; and
   diesters comprising between 17 and 40 carbon atoms in total, and in particular diesters with formula R′COOR′₂OOCR′₃ wherein R′₁ and R′₃ each represent the residue of a linear or branched fatty acid (preferably linear) including from 4 to 15 carbon atoms, and R′₂ represents a hydrocarbon chain, particularly linear, containing 2 to 10 carbon atoms;
plant-based hydrocarbon-based oils;
linear or branched alkanes having from 15 to 19 carbon atoms; and
mixtures thereof.

Preferably, the non-volatile hydrocarbon-based oil according to the invention is selected from:
C₁₀-C₂₆ alcohols, preferably monoalcohols:

More particularly, the C₁₀-C₂₆ alcohols are optionally saturated, optionally branched, and comprise from 10 to 26 carbon atoms.

Preferably, the C₁₀-C₂₆ alcohols are fatty alcohols, preferably branched when they comprise at least 16 carbon atoms.

As examples of fatty alcohols that can be used according to the invention, mention can be made of linear or branched fatty alcohols of synthetic origin, or natural origin such as for example alcohols from plant substances (coconut, palm kernel, palm, etc.) or animal substances (tallow, etc.).

Of course, other long-chain alcohols can also be used, such as for example ether-alcohols or so-called Guerbet alcohols.

Finally, certain more or less long cuts of alcohols of natural origin, such as for example coco (C₁₂ to C₁₆) or tallow (C₁₆ to C₁₈) or compounds of the diol or cholesterol type, can also be used.

Preferably a fatty alcohol comprising from 10 to 24 carbon atoms, and more preferably from 12 to 22 carbon atoms, is used.

As particular examples of fatty alcohols that can be used preferably, mention can be made in particular of lauric alcohol, isostearyl alcohol, cetearyl alcohol, oleic alcohol, 2-butyloctanol, 2-undecyl pentadecanol, 2-hexyldecylic alcohol, isocetylic alcohol, octyldodecanol and mixtures thereof, preferably cetearyl alcohol and/or octyldodecanol.
ester oils having between 17 and 40 carbon atoms selected from:
monoesters comprising between 17 and 40 carbon atoms in total, in particular monoesters, having formula R₁COOR₂ wherein R₁ is the residue of a linear or branched or aromatic fatty acid including from 4 to 40 carbon atoms, optionally saturated, and R₂ is a hydrocarbon chain in particular branched containing from 3 to 40 carbon atoms with the condition that R₁+R₂ is greater than or equal to 17, such as for example Purcellin oil (cetostearyl octanoate), cetearyl isononanoate, isononyl isononanoate, C₁₂ to C₁₅ alcohol benzoate, 2-ethyl hexyl palmitate, octyldodecyl neopentanoate, octyl-2-dodecyl stearate, octyl-2-dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, 2-ethyl-hexyl palmitate, 2-hexyl-decyl laurate, 2-octyl-decyl palmitate, 2-octyldodecyl myristate.

Preferably, these are esters having formula R₁COOR₂ wherein R₁ is the residue of a linear or branched fatty acid including from 7 to 40 carbon atoms and R₂ is a hydrocarbon chain in particular branched containing from 3 to 30 carbon atoms, with R₁ and R₂ being such that R₁+R₂ is greater than or equal to 17.

As preferred monoesters, mention can be made of cetearyl isononanoate and/or isopropyl palmitate.
diesters comprising between 17 and 40 carbon atoms in total, and in particular diesters with formula R′₁COOR′COOCR′₃ wherein R′₁ and R′₃ each represent the residue of a linear or branched fatty acid (preferably linear) including from 4 to 15 carbon atoms, and R′₂ represents a hydrocarbon chain, particularly linear, containing 2 to 10 carbon atoms. Preferably R′₁ and R′₃ are identical. As an example, mention can be made of 1,3-propanediol dicaprylate. Such a diester is marketed particularly under the name DUB Zenoat by STEARINERIES DUBOIS.
plant-based hydrocarbon-based oils such as fatty acid liquid triglycerides (liquid at ambient temperature), in particular fatty acids having from 7 to 40 carbon atoms, such as heptanoic or octanoic acid triglycerides or jojoba oil; in particular, mention can be made of saturated triglycerides such as caprylic/capric triglyceride and mixtures thereof, for example such as the one sold under the reference Myritol 318 by Cognis, glycerol triheptanoate, glycerin trioctanoate, C₁₈₋₃₆ acid triglycerides (such as those sold under the reference DUB TGI 24 by Stearineries Dubois), and unsaturated triglycerides such as castor oil, olive oil, ximenia oil, pracaxi oil.
linear or branched alkanes having from 15 to 19 carbon atoms:

As an example of alkanes suitable for the invention, mention may be made of mixtures of alkanes with between 15 and 19 carbon atoms with a molecular weight of between 200 g/mol and 250 g/mol.

Preferably, the alkanes are branched.

Preferably, the alkanes are of plant origin.

Preferably, the mixture used comprises 95 to 99% of C15-C19 branched alkanes by weight with respect to the total weight of the mixture, and 1 to 5% by weight of C12-C14 and C20-C26 alkanes with respect to the total weight of the mixture. Such a mixture preferably has a molecular weight of about 216 g/mol. Such a mixture is marketed by Seppic under the reference Emogreen L15.

Alternatively and preferably, the mixture used comprises 95 to 99% of the mixture of C15-C19 branched alkanes by weight with respect to the total weight of the mixture, and 1 to 5% of C12-C14 and C20-C26 alkanes by weight with respect to the total weight of the mixture. Such a mixture preferably has a molecular weight of about 248 g/mol. Such a mixture is marketed by Seppic under the reference Emogreen L19.

Alternatively, a mixture is used containing 10% by weight of C15-C19 branched alkanes with respect to the total weight of the mixture, and 1 to 5% by weight of C12-C14 and C20-C26 alkanes with respect to the total weight of the mixture. Such a mixture is marketed by Seppic under the reference Emosmart L19.
and mixtures thereof.

Preferably, the non-volatile hydrocarbon-based oil according to the invention is selected from C₁₀-C₂₆ alcohols, monoesters comprising between 17 and 40 carbon atoms in total, in particular monoesters with formula R₁COOR₂ wherein R₁ is the residue of a linear or branched or aromatic fatty acid including from 4 to 40 carbon atoms, optionally saturated, and R₂ is a hydrocarbon chain in particular branched containing from 3 to 40 carbon atoms, with the condition that R₁+R₂ is greater than or equal to 17, and mixtures thereof.

More preferably, the non-volatile hydrocarbon-based oil according to the invention is selected from cetearyl isononanoate, isopropyl palmitate, cetearyl alcohol, octyldodecanol and mixtures thereof.

Preferably, the composition comprises between 2% and 40%, preferably between 3% and 35%, preferably between 4% and 30% by weight of non-volatile hydrocarbon-based oil with respect to the total weight of the composition.

### Cupuaçu Butter

The composition according to the invention comprises at least one cupuaçu butter.

Cupuaçu butter is a butter of plant origin. It is considered as a pasty fatty substance.

For the purposes of the invention, the term “pasty fatty substance” refers to a lipophilic fatty compound having a reversible solid/liquid change of state, having an anisotropic crystalline organization in the solid state, and including a liquid fraction and a solid fraction at a temperature of 23° C.

In other words, the initial melting point of the pasty compound may be less than 23° C. The liquid fraction of the pasty compound measured at 23° C. may represent 9 to 97% by weight of the compound. This liquid fraction at 23° C. preferably represents between 15 and 85%, more preferably between 40 and 85% by weight.

The melting point of a solid fatty substance can be measured using a differential scanning calorimeter (DSC), for example the calorimeter sold under the trade name “DSC Q100” by TA Instruments with “TA Universal Analysis” software, according to the protocol defined hereinabove.

The liquid fraction by weight of the pasty compound at 23° C. is more particularly equal to the ratio of the enthalpy of fusion consumed at 23° C. to the enthalpy of fusion of the pasty compound.

The enthalpy of fusion of the pasty compound is the enthalpy consumed by the compound to change from the solid state to the liquid state. The pasty compound is said to be in the solid state when the entire mass thereof is in solid crystalline form. The pasty compound is said to be in the liquid state when the entire mass thereof is in liquid form.

The enthalpy of fusion of the pasty compound is in particular equal to the area under the curve of the thermogram obtained using a differential scanning calorimeter. The enthalpy of fusion of the pasty compound is the quantity of energy required to change the compound from the solid state to the liquid state. It is expressed in J/g.

The enthalpy of fusion consumed at 23° C. is the quantity of energy required by the sample to change from the solid state to the state presented at 23° C. consisting of a liquid fraction and a solid fraction.

Cupuaçu (THEOBROMA GRANDIFLORUM) is native plant of the Amazon rainforest, rich in fatty acids.

Cupuaçu butter (INCI name: THEOBROMA GRANDIFLORUM SEED BUTTER) is marketed particularly under the name Rain forest RF3410 by BERACA INGR. NATURAIS (CLARIANT).

Preferably, the composition comprises between 0.1% and 40%, preferably between 0.5% and 30%, preferably between 1% and 20%, more preferably between 1.5% and 10% by weight of cupuaçu butter with respect to the total weight of the composition.

Preferably, the composition according to the invention also comprises at least one butter of plant origin other than cupuaçu butter. Preferably, the composition according to the invention also comprises at least one jojoba butter (INCI name: SIMMONDSIA CHINENSIS (JOJOBA) BUTTER/SIMMONDSIA CHINENSIS BUTTER).

Preferably, the composition comprises between 0.1% and 10%, preferably between 0.3% and 8%, preferably between 0.5% and 5%, by weight of butter of plant origin other than cupuaçu butter, preferably jojoba butter, with respect to the total weight of the composition.

### Wax

The composition according to the invention also comprises at least one wax.

“Wax” refers to a lipophilic compound, which is solid at ambient temperature (25° C.), having a reversible solid/liquid change of state, having a melting point in particular greater than or equal to 30° C. and less than or equal to 90° C., more particularly less than or equal to 80° C., and preferably less than or equal to 70° C. The melting point of a solid fatty substance can be measured using a differential scanning calorimeter (DSC), for example the calorimeter sold under the trade name “DSC Q100” by TA Instruments with “TA Universal Analysis” software.

The measurement protocol is as follows:

A sample of solid fat of about 5 mg is placed in a “sealed aluminum capsule” crucible. When the solid fat is soft (pasty fat), the sample is subjected to a first temperature rise from 20° C. to 80° C., at a heating rate of 2° C./minute, and to 80° C., then left at isotherm of 80° C. for 20 minutes, then is cooled from 80° C. to −80° C. at a cooling rate of 2° C./minute, and finally subjected to a second temperature rise from −80° C. to 20° C. at a heating rate of 2° C./minute.

The value of the melting temperature of the solid fat is the value of the top of the most endothermic peak of the fusion curve observed, representing the variation in the difference in power absorbed as a function of the temperature. In particular, the polar waxes implemented in the composition according to the invention, have a melting temperature greater than 30° C. and better greater than 45° C.

More particularly, the wax is selected from polar waxes and non-polar waxes, particularly hydrocarbon-based. The polar waxes are particularly ester hydrocarbon waxes, alcohol hydrocarbon waxes, silicone waxes, as well as mixtures thereof.

The term “hydrocarbon wax” refers to a wax essentially formed, or consisting, of carbon and hydrogen atoms, and optionally oxygen, nitrogen atoms, and containing no silicon or fluorine. It may contain alcohol, ester, ether, carboxylic acid, amine and/or amide groups.

The term “ester wax” refers according to the invention to a wax that comprises at least one ester function. The ester waxes can furthermore be hydroxylated.

The term “alcohol wax” refers according to the invention to a wax that comprises at least one alcohol function, i.e., that comprises at least one free hydroxyl (OH) group. The additional alcohol wax does not comprise in particular any ester function.

The term “silicon oil” refers to an oil comprising at least one silicon atom and particularly comprising Si—O groups.

### Ester Waxes

The following can in particular be used as an ester wax:
ester waxes such as those selected from:
i) the waxes having formula R₁COOR₂ wherein R₁ and R₂ are aliphatic linear, branched or cyclic chains of which the number of atoms varies from 10 to 50, that can contain a heteroatom in particular oxygen, and of which the melting point temperature varies from 30 to 120° C., preferably from 30 to 100° C. In particular it is possible to use as ester wax a C₂₀-C₄₀ (hydroxystearyloxy)alkyl stearate (with the alkyl group comprising from 20 to 40 carbon atoms), alone or in a mixture or a C20-C40 alkyl stearate. Such waxes are particular sold under the names “Kester Wax K 82 P®”, “Hydroxypolyester K 82 P®”, “Kester Wax K 80 P®”, or “KESTER WAX K82H” by KOSTER KEUNEN. Mixtures of C14-C18 carboxylic acid esters and of alcohols such as the “Cetyl Ester Wax 814” product from KOSTER KEUNEN, “SP Crodamol MS MBAL”, “Crodamol MS PA” from CRODA, “Miraceti” from LASERSON can also be used.

A montanate (octacosanoate) of glycol and of butylene glycol can also be used such as the LICOWAX KPS FLAKES wax (INCI name: glycol montanate) marketed by Clariant;
ii) di-(trimethylol-1,1,1 propane) tetrastearate, sold under the name Hest 2T-4S® by HETERENE;
iii) diester waxes of a carboxylic diacid having general formula R³—(—OCO—R⁴—COO—R⁵), wherein R³ and R⁵ are identical or different, preferably identical and is a C₄-C₃₀ alkyl group (alkyl group comprising from 4 to 30 carbon atoms) and R⁴ is a C₄-C₃₀ linear or branched aliphatic group (alkyl group comprising from 4 to 30 carbon atoms) and which can optionally contain one or several unsaturations. Preferably, the C₄-C₃₀ aliphatic group is linear and unsaturated;
iv) the waxes obtained by catalytic hydrogenation of animal or plant oils that in particular have C₈-C₃₂ linear or branched fat chains, for example such as hydrogenated jojoba oil, hydrogenated sunflower oil, hydrogenated castor oil, hydrogenated coconut oil, as well as the waxes obtained by hydrogenating esterified castor oil with cetyl alcohol, such as those sold under the names Phytowax ricin 16L64® and 22L73® by SOPHIM. Such waxes are described in application FR-A-2792190. As waxes obtained by hydrogenating esterified olive oil with stearyl alcohol, mention can be made of those sold under the name “PHYTOWAX Olive 18 L 57”;
v) waxes of plant origin, such as beeswax, synthetic beeswax, carnauba wax, candelilla wax, rice bran wax, Ouricury wax, Alfa wax, berry wax, shellac wax, cork fiber wax, sugarcane wax, Japan wax, sumac wax, montan wax, Orange and Lemon waxes, Bay leaf wax, hydrogenated Jojoba wax, sunflower wax, in particular refined;
vi) Mention can also be made of hydrocarbon, polyoxyalkylene or polyglycerol waxes, natural or synthetic, of animal or plant origin; the number of oxyalkylene units (C2-C4) can vary from 2 to 100, the number of glycerol units can vary from 1 to 20. As examples, mention can be made of polyoxyethylene beeswax, such as PEG-6 beeswax, PEG-8 beeswax; polyoxyethylene carnauba waxes, such as PEG-12 carnauba; lanolin waxes, optionally hydrogenated, polyoxyethene or polyoxypropylene, such as PEG-30 lanolin, PEG-75 lanolin; PPG-5 lanolin wax glyceride; polyglycerol beeswax, in particular polyglyceryl-3 Beewax, the Acacia Decurrens/Jojoba/Sunflower Seed Wax/Polyglyceryl-3 Esters mixture, polyglycerol plant waxes such as mimosa, jojoba, sunflower waxes, and mixtures thereof (Acacia Decurrens/Jojoba/Sunflower Seed Wax Polyglyceryl-3 Esters; vii) Waxes corresponding to partial or total esters, preferably total, of a C₁₆-C₃₀ carboxylic, saturated, optionally hydroxylated, with glycerol. The term total esters means that all of the hydroxyl functions of the glycerol are esterified. As an example, mention can be made of trihydroxystearin (or glyceryl trihydroxystearate), tristearin (or glyceryl tristearate), tribehenin (or glyceryl tribehenate), alone or in a mixture. Among the suitable compounds, mention can be made of glycerol triesters and of 12-hydroxystearic acid or hydrogenated castor oil, such as for example Thixcin R, Thixcin E, sold by Elementis Specialties;
viii) as well as mixtures thereof.

### Alcohol Waxes

As for alcohol wax, mention can be made of alcohols, preferably linear, preferably saturated, comprising from 16 to 60 carbon atoms, of which the melting point is between 25° C. and 90° C. As examples of alcohol wax, mention can be made of stearyl alcohol, cetyl alcohol, myristyl alcohol, palm alcohol, behenic alcohol, erucic alcohol, arachidyl alcohol, or mixtures thereof.

### Silicone Waxes

As silicone wax, mention can be made for example of mixtures comprising a compound of the C30-45 Alkyldimethylsilyl Polypropylsilsesquioxane type (INCI name), for example the Dow Corning product SW-8005 C30 Resin Wax sold by Dow Corning. Mention can also be made of mixtures comprising a compound of the C30-45 Alkyl Methicone (INC name) type, such as for example the Dow Corning® product AMS-C30 Cosmetic Wax. Mention can also be made of silicone beeswax.

For the purposes of the invention, “non-polar hydrocarbon wax” refers to a wax that comprises only carbon or hydrogen atoms in its structure. In other terms, such a wax is free of other atoms, in particular heteroatoms such as for example nitrogen, oxygen, silicon. For the purposes of illustration of non-polar waxes suitable for the invention, mention can in particular be made of hydrocarbon waxes such as microcrystalline waxes, paraffin waxes, ozokerite, polymethylene waxes, polyethylene waxes, waxes obtained by Fischer-Tropsch synthesis, microwaxes in particular polyethylene.

Preferably, the composition according to the invention comprises at least one polar wax, preferably selected from waxes of plant origin, such as beeswax, synthetic beeswax, carnauba wax, candelilla wax, rice bran wax, Ouricury wax, Alfa wax, berry wax, shellac wax, cork fiber wax, sugarcane wax, Japan wax, sumac wax, montan wax, Orange and Lemon waxes, Bay leaf wax, hydrogenated Jojoba wax, and sunflower wax, in particular refined.

Preferably, the wax content is between 0.05 and 5% by weight with respect to the total weight of the composition, preferably between 0.1 and 3%, preferably between 0.3 and 2% by weight.

### Aqueous Phase

The composition according to the invention comprises a physiologically acceptable aqueous medium which forms the continuous aqueous phase. “Physiologically acceptable” refers to a medium compatible with keratin materials.

The composition according to the invention preferably comprises an aqueous medium comprising water and optionally an organic solvent soluble in water, at 25° C., selected for example from linear or branched C2-C4 alkanols, such as ethanol and isopropanol, propanol, butanol; polyols and mixtures thereof.

For the purposes of the invention, polyol refers to a hydrocarbon chain including at least 2 carbon atoms, preferably 2 to 50 carbon atoms, preferably 4 to 20 carbon atoms, preferably having 2 to 10 carbon atoms, and preferably having 2 to 6 carbon atoms, and carrying at least two hydroxy groups. The polyols used in the present invention can have an average molecular mass by weight of less than or equal to 1000, and preferably between 90 and 500. The polyol can be a natural or synthetic polyol. The polyol can have a linear, branched or cyclic molecular structure.

This polyol can be selected from glycerin and derivatives thereof, and glycols and derivatives thereof. The polyol can be selected from the group composed of glycerin, diglycerin, polyglycerin, diethylene glycol, propylene glycol, dipropylene glycol, butylene glycol, pentylene glycol, hexylene glycol, 1,3-propanediol, 1,5-pentanediol, octane 1,2-diol, polyethyleneglycols, particularly having from 5 to 50 ethylene oxide groups, and sugars such as sorbitol, and mixtures thereof. More particularly, the polyol is glycerin.

Preferably, the composition comprises water, at least one linear or branched C2-C4 alkanol and at least one polyol.

Preferably, the composition comprises at least 50% by weight of water with respect to the total weight of the composition, preferably at least 55%, preferably at least 60% by weight. Preferably, the composition comprises from 50 to 95% by weight of water with respect to the total weight of the composition, preferably from 55 to 80% by weight.

The amount of organic solvent(s) can range for example from 0.1 to 30% by weight, preferably from 1 to 25% by weight, still better from 5 to 20% by weight with respect to the total weight of the composition.

The composition can also comprise at least one polymer, and particularly a hydrophilic polymer. Such a polymer is preferably a water-soluble or water-dispersible AMPS® polymer.

The water-soluble or water-dispersible AMPS® polymers preferably have a molar mass ranging from 50,000 g/mole to 10,000,000 g/mole, preferably from 80,000 g/mole to 8,000,000 g/mole, and more preferably from 100,000 g/mole to 7,000,000 g/mole.

As water-soluble or water-dispersible AMPS homopolymers suitable for the invention, mention may be made for example of optionally cross-linked sodium 2-acrylamido-2-methyl propane sulfonate acid polymers such as that used in the commercial product SIMULGEL 800 (CTFA name: Sodium Polyacryloyldimethyl Taurate), cross-linked polymers of ammonium 2-acrylamido-2-methyl propane sulfonate acid (INCI name: AMMONIUM POLYACRYLDIMEHYLTAURAMIDE) such as the product sold under the trade name HOSTACERIN AMPS® by Clariant.

As water-soluble or water-dispersible AMPS copolymers according to the invention, mention may be made for example of:
cross-linked sodium acrylamide/acrylamido-2-methyl propane sulfonate copolymers such as those used in the commercial product SEPIGEL 305 (CTFA name: 5 3028758 57 POLYACRYLAMIDE/C13-C14 ISOPARAFFIN/LAURETH-7) or that used in the commercial product sold under the name SIMULGEL 600 (CTFA name: ACRYLAMIDE/SODIUM ACRYLOYLDIMETHYLTAURATE/ISOHEXADECANE/POLYSORBATE-80) by SEPPIC;
copolymers of AMPS® and vinylpyrrolidone or vinylformamide such as that used in the commercial product sold under the trade name ARISTOFLEX AVC® by CLARIANT (CTFA name: AMMONIUM ACRYLOYLDIMETHYLTAURATE/VP COPOLYMER) but neutralized with soda or potash;
copolymers of AMPS® and sodium acrylate, such as for example the AMPS/sodium acrylate copolymer such as that used in the commercial product sold under the trade name SIMULGEL EG® by SEPPIC or under the trade name SEPINOV EM as (CTFA name: HYDROXYETHYL ACRYLATE/SODIUM ACRYLOYLDIMETHYL TAURATE COPOLYMER);
copolymers of AMPS® and hydroxyethyl acrylate, such as for example the AMPS®/hydroxyethyl acrylate copolymer like that used in the commercial product sold under the trade name SIMULGEL NS® by SEPPIC (CTFA name: HYDROXYETHYL ACRYLATE/SODIUM ACRYLOYLDIMETHYLTAURATE COPOLYMER (AND) SQUALANE (AND) POLYSORBATE 60) or as the product marketed under the name SODIUM ACRYLAMIDO-2-METHYLPROPANE SULFONATE/HYDROXYETHYLACRYLATE COPOLYMER such as the commercial product SEPINOV EMT 10 (INCI name: HYDROXYETHYL ACRYLATE/SODIUM ACRYLOYLDIMETHYL TAURATE COPOLYMER);
copolymers of AMPS® and ethoxylated cetearyl methacrylate, optionally cross-linked, such as Aristoflex® HMS, that has the name AMPS copolymer/ethoxylated cetearyl methacrylate (25 EO) 80/20, cross-linked by trimethylolpropane triacrylate (TMPTA) or Ammonium Acryloyldimethyltaurate/Steareth-25 methacrylate crosspolymer with INCI name.

The aqueous phase of the composition can also comprise at least one additional surfactant other than the non-ionic surfactants. Preferably, the additional surfactant is selected from:
b) anionic surfactants such as:
   polyoxyethylenated fatty acid salts and particularly those derived from alkaline salts, and mixtures thereof;
   phosphoric esters and their salts such as “DEA oleth-10 phosphate” (Crodafos N 10N from CRODA) or monopotassium monocetyl phosphate (Amphisol K from Givaudan);
   sulfosuccinates such as “Disodium PEG-5 citrate lauryl sulfosuccinate” and “Disodium ricinoleamido MEA sulfosuccinate”;
   alkylethersulfates such as sodium lauryl ether sulfate;
   isethionates;
   acylglutamates such as “Disodium hydrogenated tallow glutamate” (AMISOFT HS-21 R® marketed by AJINOMOTO) and sodium stearoyl glutamate (AMISOFT HS-11 PF® marketed by AJINOMOTO) and mixtures thereof;
   derivatives of soybeans such as potassium soyate;
   citrates, such as Glyceryl stearate citrate (Axol C 62 Pellets from Degussa);
   derivatives of proline, such as Sodium palmitoyl proline (Sepicalm VG from Seppic), or the Mixture of Sodium palmitoyl sarcosinate, Magnesium palmitoyl glutamate, palmitic acid and Palmitoyl proline (Sepifeel One from Seppic);
   lactylates, such as Sodium stearoyl lactylate (Akoline SL from Karlshamns AB);
   sarcosinates, such as sodium palmitoyl sarcosinate (Nikkol sarcosinate PN) or the mixture of Stearoyl sarcosine and Myristoyl sarcosine 75/25 (Crodasin SM from Croda);
   sulfonates, such as Sodium C14-17 alkyl see sulfonate (Hostapur SAS 60 from Clariant);
   glycinates, such as sodium cocoyl glycinate (Amilite GCS-12 from Ajinomoto);
   C16-C30 fatty acid salts in particular those derived from amines, such as triethanolamine stearate and/or amino-2-methyl-2-propane di-ol-1,3 stearate;
b) amphoteric surfactants, such as N-acyl-aminoacids such as N-alkyl-aminoacetates, disodium cocoamphodiacetate and amine oxides such as stearamine oxide or even silicone surfactants such as dimethicone copolyol phosphates such as the one sold under the trade name PECOSIL PS 100® by PHOENIX CHEMICAL;
and mixtures thereof.

The composition according to the invention also comprises an oily phase dispersed in the aqueous phase.

The composition can comprise, besides the non-volatile hydrocarbon-based oil and cupuaçu butter, oils other than the non-volatile hydrocarbon-based oil, organopolysiloxane elastomers or mixtures thereof.

Among the oils, mention may be made of silicone oils, volatile hydrocarbon-based oils, and mixtures thereof.

The composition can also comprise at least one organopolysiloxane elastomer.

“Organopolysiloxane elastomer” or “silicone elastomer” refers to a soft deformable organopolysiloxane, having viscoelastic properties and particularly the consistency of a sponge or a soft sphere. The modulus of elasticity thereof is such that this material resists deformation and has a limited extendibility and contractability. This material is capable of recovering the original shape thereof after stretching. It consists more particularly of a cross-linked organopolysiloxane elastomer.

Thus, the organopolysiloxane elastomer can be obtained by addition cross-linking reaction of diorganopolysiloxane that contains at least one silicon-bonded hydrogen and diorganopolysiloxane having silicon-bonded ethylenic unsaturated groups, in particular in the presence of a platinum catalyst; or by condensation dehydrogenation cross-linking reaction between a hydroxyl-terminated diorganopolysiloxane and a diorganopolysiloxane that contains at least one silicon-bonded hydrogen, in particular in the presence of an organotin; or by condensation cross-linking reaction of a hydroxyl-terminated diorganopolysiloxane and a hydrolysable organopolysilane; or by thermal cross-linking of organopolysiloxane, in particular in the presence of an organic peroxide catalyst; or by organopolysiloxane cross-linking by high-energy radiation such as gamma rays, ultraviolet rays, electron beams.

Preferably, the organopolysiloxane elastomer is obtained by addition cross-linking reaction (A) of diorganopolysiloxane that contains at least two silicon-bonded hydrogens, and (B) of diorganopolysiloxane having at least two silicon-bonded ethylenic unsaturated groups, in particular in the presence (C) of a platinum catalyst, as for example described in application EP-A-295886.

In particular, the organopolysiloxane elastomer can be obtained by reaction of dimethylvinylsiloxy-terminated dimethylpolysiloxane and of trimethylsiloxy-terminated methylhydrogenopolysiloxane, in the presence of a platinum catalyst.

The compound (A) is the basic reagent for the formation of organopolysiloxane elastomer and the crosslinking takes place via addition reaction of the compound (A) with the compound (B) in the presence of the catalyst (C).

The compound (A) is in particular an organopolysiloxane having at least two hydrogen atoms bonded to separate silicon atoms in each molecule. The compound (A) can have any molecular structure, in particular a linear chain or branched chain structure or a cyclical structure.

The compound (A) can have a viscosity at 25° C. ranging from 1 to 50,000 centistokes, in particular in order to be miscible with the compound (B).

The organic groups bonded to the silicon atoms of the compound (A) can be alkyl groups such as methyl, ethyl, propyl, butyl, octyl; substituted alkyl groups such as 2-phenylethyl, 2-phenylpropyl, 3,3,3-trifluoropropyl; aryl groups such as phenyl, tolyl, xylyl; substituted aryl groups such as phenylethyl; and substituted monovalent hydrocarbon groups such as an epoxy group, a carboxylate ester group, or a mercapto group.

The compound (A) can thus be selected from trimethylsiloxy-terminated methylhydrogenopolysiloxanes, trimethylsiloxy-terminated dimethylsiloxane-methylhydrogenosiloxane copolymers, dimethylsiloxane-methylhydrogenosiloxane cyclical polymers.

The compound (B) is advantageously a diorganopolysiloxane that has at least two lower alkenyl groups (for example in C2-C4); the lower alkenyl group can be selected from the vinyl, allyl, and propenyl groups. These lower alkenyl groups can be located at any position of the organopolysiloxane molecule but are preferably located at the ends of the organopolysiloxane molecule. The organopolysiloxane (B) can have a branched chain, linear chain, cyclical or network structure but the linear chain structure is preferred. The compound (B) can have a viscosity ranging from the liquid state to the gum state. Preferably, the compound (B) has a viscosity of at least 100 centistokes at 25° C.

Besides the alkenyl groups cited above, the other organic groups bonded to the silicon atoms of the compound (B) can be alkyl groups such as methyl, ethyl, propyl, butyl or octyl; substituted alkyl groups such as 2-phenylethyl, 2-phenylpropyl or 3,3,3-trifluoropropyl; aryl groups such as phenyl, tolyl or xylyl; substituted aryl groups such as phenylethyl; and substituted monovalent hydrocarbon groups such as an epoxy group, a carboxylate ester group, or a mercapto group.

The organopolysiloxanes (B) may be selected from methylvinylpolysiloxanes, methylvinylsiloxane-dimethylsiloxane copolymers, dimethylvinylsiloxy-terminated dimethylpolysiloxanes, dimethylvinylsiloxy-terminated dimethylsiloxane-methylphenylsiloxane copolymers, dimethylvinylsiloxy-terminated dimethylsiloxane-diphenylsiloxane-methylvinylsiloxane copolymers, trimethylsiloxy-terminated dimethylsiloxane-methylvinylsiloxane copolymers, trimethylsiloxy-terminated dimethylsiloxane-methylphenylsiloxane-methylvinylsiloxane copolymers, dimethylvinylsiloxy-terminated methyl(3,3,3-trifluoropropyl)polysiloxane, and dimethylvinylsiloxy-terminated dimethylsiloxane-methyl(3,3,3-trifluoropropyl)siloxane copolymers.

In particular, the organopolysiloxane elastomer can be obtained by reaction of dimethylvinylsiloxy-terminated dimethylpolysiloxane and of trimethylsiloxy-terminated methylhydrogenopolysiloxane, in the presence of a platinum catalyst.

Advantageously, the sum of the number of ethylenic groups per molecule of the compound (B) and the number of hydrogen atoms bonded to silicon atoms per molecule of the compound (A) is at least 5.

It is advantageous that the compound (A) be added in a quantity such that the molecular ratio between the total quantity of hydrogen atoms bonded to silicon atoms in the compound (A) and the total quantity of all of the ethylenic unsaturated groups in the compound (B) be in the range from 1.5:1 to 20:1.

The compound (C) is the catalyst of the crosslinking reaction, and is in particular chloroplatinic acid, chloroplatinic-olefin acid complexes, chloroplatinic-alkenylsiloxane acid complexes, chloroplatinic-diketone acid complexes, black platinum, and supported platinum.

The catalyst (C) is preferably added from 0.1 to 1000 parts by weight, and better from 1 to 100 parts by weight, as a clean platinum metal for 1000 parts by weight of the total quantity of the compounds (A) and (B).

The organopolysiloxane elastomer is advantageously an emulsifying elastomer.

The term “emulsifying” defines organopolysiloxane elastomers containing a hydrophilic chain, and in particular containing polyoxyalkylene structural units (particularly polyoxyethylene or polyoxypropylene) and/or polyglyceryl structural units.

In particular, the organopolysiloxane elastomer used in the present invention is a Dimethicone/Polyglycerin-3 Crosspolymer-3 (INCI name).

The organopolysiloxane elastomer particles can be carried in the form of a gel consisting of an elastomeric organopolysiloxane included in at least one hydrocarbon oil and/or a silicone oil. In these gels, the organopolysiloxane particles are often non-spherical particles.

Emulsifying elastomers are particularly described in the patents EP 242 219, EP 285 886, EP 765 656 and in the application JP-A-61-194009. The organopolysiloxane elastomer is generally in the form of a gel, a paste or a powder but advantageously in the form of a gel wherein the organopolysiloxane elastomer is dispersed in a silicone oil that is linear (dimethicone) or cyclic (e.g.: cyclopentasiloxane), advantageously in a linear silicone oil.

As emulsifying elastomer, that sold under the trade name “KSG-710” by Shin Etsu can more particularly be used.

According to a particular embodiment, an organopolysiloxane elastomer gel dispersed in a silicon oil selected from a non-exhaustive list comprising cyclopentadimethylsiloxane, dimethicones, dimethylsiloxanes, methyl trimethicone, phenylmethicone, phenyldimethicone, phenyltrimethicone, and cyclomethicone, preferably a linear silicone oil selected from polydimethylsiloxanes (PDMS) or dimethicones having a viscosity at 25° C. ranging from 1 to 500 is at 25° C., optionally modified by aliphatic groups, optionally fluorinated, or by functional groups such as hydroxyl, thiol and/or amine groups, is used.

The total quantity in the cosmetic composition according to the present invention of organopolysiloxane elastomer(s) represents an active substance content of 0.1% to 7% by weight, in particular from 0.2% to 5% by weight, preferably from 0.6% to 4% by weight with respect to the total weight of the composition.

The composition according to the invention can also comprise any additional ingredient well known to a person skilled in the art, such as preservatives, perfumes, fillers, vitamins, active agents, UV filters and/or sequestering agents.

Preferably, the sequestering agent is selected from ethylene diamine disuccinic acid (EDDS) and salts thereof. The ethylene diamine disuccinic acid is the compound of formula (II):

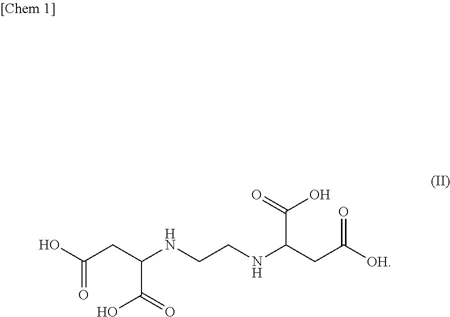 [SMILES: O=C(O)CC(NCCNC(CC(=O)O)C(=O)O)C(=O)O]

Preferably, the ethylene diamine disuccinic acid salt is selected from alkali metal salts, such as potassium and sodium salts, ammonium salts, and amine salts. The alkali metal salts of ethylenediamine disuccinic acid are more specifically preferred.

Preferably, the ethylene diamine disuccinic acid salt used according to the invention is trisodium ethylene diamine disuccinate. Such a compound is for example that marketed under the trade name Natrlquest® E30 by Innospec Active Chemicals (dispersion of the compound at 37% by weight of active substance in water), or that marketed under the trade name Octaquest E30® by Octel Performance Chemicals.

The composition according to the invention preferably comprises a quantity of sequestering agent between 0.01% and 1% by weight with respect to the total weight of the composition, preferably ranging from 0.05% to 0.8% by weight, and more preferably ranging from 0.06% to 0.7% by weight.

The invention also relates to a cosmetic method for cleansing keratin materials, preferably the skin, comprising the application of a composition according to the invention on said keratin materials.

The present invention also relates to the cosmetic use of a composition according to the invention as an anti-aging composition.

Concrete, yet non-limiting, examples, illustrating the invention, will now be provided.

In the examples, the temperature is ambient temperature (20° C.) expressed in degrees Celsius unless mentioned otherwise, and the pressure is atmospheric pressure, unless mentioned otherwise.

In the examples, quantities of the ingredients of the compositions are given as a % by weight relative to the total weight of the composition (% w/w).

### EXAMPLES

In the examples, the compositions are prepared with the ingredients mentioned in the tables hereinbelow, according to the following protocol:
The ingredients of the oily phase A (i.e., oils, jojoba butter and surfactants);
Heat to 75° C.;
Disperse until melted and fully solubilized;
The gelling agents and cupuaçu butter are added;
Then emulsified: the aqueous phase B at 75° C. is poured onto the oily phase, then disperse to obtain an emulsion and cooled.
Phase C: The previously mixed active agents are introduced with some of the water followed by the perfume at 40° C., and disperse until an acceptable emulsion is obtained.
Phase D: The silicone elastomer at 35° C. is then introduced.
Disperse; at 30° C., ethanol is introduced (Phase E).

Formula A is according to the invention, whereas formula B is comparative.

TABLE 1

| | | Formula A | Formula B |
|---|---|---|---|
| | | (invention) | (comparative) |
| Ingredient | Phase | (% w/w) | (% w/w) |
| GLYCERYL STEARATE (and) | A | 2.5 | 2.5 |
| PEG-100 STEARATE | | | |
| (Arlacel 65 by Croda) | | | |
| ORBIGNYA OLEIFERA | A | 0.9 | 0.9 |
| SEED OIL | | | |
| (Babassu seed oil) | | | |
| ISOPROPYL PALMITATE | A | 1.2 | 1.2 |
| Beeswax | A | 0.5 | — |
| DIMETHICONE | A | 1.4 | 1.4 |
| POTASSIUM CETYL | A | 0.2 | 0.2 |
| PHOSPHATE | | | |
| (Amphisol K by Givaudan) | | | |
| CETEARYL ISONONANOATE | A | 2.5 | 2.5 |
| CETEARYL ALCOHOL (and) | A | 4 | 4 |
| CETEARYL GLUCOSIDE | | | |
| (Montanov 68) | | | |
| PENTAERYTHRITYL | A | 0.11 | 0.11 |
| TETRA-DI-T-BUTYL | | | |
| HYDROXYHYDRO- | | | |
| CINNAMATE | | | |
| Jojoba butter | A | 0.6 | — |
| (SIMMONDSIA | | | |
| CHINENSIS BUTTER) | | | |
| HYDROXYETHYL | A | 1 | 1 |
| ACRYLATE/SODIUM | | | |
| ACRYLOYLDIMETHYL | | | |
| TAURATE COPOLYMER | | | |
| (SEPINOV EM by Seppic) | | | |
| Cupuaçu butter | A | 3 | — |
| (THEOBROMA | | | |
| GRANDIFLORUM | | | |
| SEED BUTTER) | | | |
| (Rain forest RF3410 | | | |
| by Beraca Ingr. | | | |
| Naturais (Clariant)) | | | |
| OCTYLDODECANOL | A | 1 | 1 |
| Preservative | B | QS | QS |
| TRISODIUM | B | 0.2 | 0.2 |
| ETHYLENEDIAMINE | | | |
| DISUCCINATE | | | |
| (Natrlquest ® E30 | | | |
| by Innospec | | | |
| Active Chemicals) | | | |
| Water | B | QS 100 | QS 100 |
| GLYCERIN | B | 5 | 5 |
| PERFUME | C | 0.25 | 0.25 |
| Active ingredients | C | QS | QS |
| DIMETHICONE (and) | D | 4 | 4 |
| DIMETHICONE/ | | | |
| POLYGLYCERIN-3 | | | |
| CROSSPOLYMER | | | |
| (KSG-710 by Shin Etsu) | | | |
| Ethanol | E | 3 | 3 |

The penetration rate of each formula is measured using the following protocol:

150 microliters of each formula is applied on the back of the hand, 1 revolution per second is performed, then the penetration time of each formula is assessed with a timer.

The optimal penetration rate is defined as follows: having measured the penetration rate of various anti-aging formulas, according to consumer ratings, it is concluded that the optimal penetration rate is between 19 and 25 seconds.

The results are as follows:

TABLE 2

| | Formula A | Formula B |
|---|---|---|
| Formula | (invention) | (comparative) |
| Volunteer 1 | 19 seconds | 17 seconds |
| Volunteer 2 | 20 seconds | 17 seconds |

As the results show, the presence of beeswax and butter in the formula A according to the invention makes it possible to obtain an optimal penetration rate, i.e., a penetration rate that it is not too quick so that the consumer has enough time to spread it on the face, but not too slow either so that the covering, nourishing and tightening effects of the formula are perceived. If the penetration rate is too quick, then the formula will not be applied homogeneously on the face and is less comfortable for the consumer, who needs to stretch it to spread it. The comparative formula B has a less optimal penetration rate.

1. A composition in the form of an oil-in-water emulsion comprising:
at least one non-ionic surfactant,
at least one non-volatile hydrocarbon-based oil,
at least one cupuaçu butter, and
at least one wax.

2. The composition according to claim 1, wherein the non-ionic surfactant is selected from fatty acid and polyethylene glycol esters, alkylC8-C30(poly)glycosides and mixtures thereof.

3. The composition according to claim 2, wherein the fatty acid and polyethylene glycol ester is a C₁₆-C₂₂ fatty acid ester including from 8 to 200 ethylene oxide units.

4. The composition according to claim 2, wherein the alkylC8-C30(poly)glycoside has the following general formula: 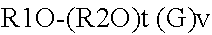
wherein R1 is a linear or branched alkyl and/or alkenyl radical including about 8 to 30 carbon atoms, an alkylphenyl radical of which the linear or branched alkyl radical includes from 8 to 24 carbon atoms; R2 is an alkylene radical including about from 2 to 4 carbon atoms; G is a sugar structural unit including from 5 to 6 carbon atoms; t denotes a value ranging from 0 to 10; and v denotes a value ranging from 1 to 15.

5. The composition according to claim 2, wherein the alkylC8-C30(poly)glycoside is selected from coco(poly)glucoside, arachidyl(poly)glucoside, Myristyl(poly)glucoside, cetylstearyl(poly)glucoside, C12-C20 alkyl(poly)glucosides, isostearyl(poly)glucoside and octyldodecyl(poly)xyloside.

6. The composition according to claim 1, which comprises a mixture of at least one fatty acid and polyethylene glycol ester and at least one alkylC8-C30(poly)glycoside.

7. The composition according to claim 1, wherein the non-volatile hydrocarbon-based oil is selected from:
C₁₀-C₂₆ alcohols;
ester oils having between 17 and 40 carbon atoms;
plant-based hydrocarbon-based oils;
linear or branched alkanes having from 15 to 19 carbon atoms; and
mixtures thereof.

8. The composition according to claim 1, wherein the non-volatile hydrocarbon-based is selected from C₁₀-C₂₆ alcohols, monoesters comprising between 17 and 40 carbon atoms in total, and mixtures thereof.

9. The composition according to claim 1, which comprises between 0.1% and 40% of cupuaçu butter with respect to the total weight of the composition.

10. The composition according to claim 1, which comprises at least one wax selected from polar waxes and non-polar waxes.

11. The composition according to claim 1, which also comprises at least one butter of plant origin other than cupuaçu butter.

12. The composition according to claim 1, which comprises a continuous aqueous phase and optionally an organic solvent soluble in water, at 25° C.

13. The composition according to claim 1, which comprises, besides the non-volatile hydrocarbon-based oil and cupuaçu butter, at least one compound selected from oils other than the non-volatile hydrocarbon-based oil, organopolysiloxane elastomers and mixtures thereof.

14. A cosmetic care method of keratin materials comprising application of a composition according to claim 1 on said keratin materials.

15. A cosmetic method of applying a composition according to claim 1 as an anti-aging composition on keratin materials.

16. The composition according to claim 4, wherein the alkylC8-C30(poly)glycoside is selected from coco(poly)glucoside, arachidyl(poly)glucoside, Myristyl(poly)glucoside, cetylstearyl(poly)glucoside, C12-C20 alkyl(poly)glucosides, isostearyl(poly)glucoside and octyldodecyl(poly)xyloside.

17. The composition according to claim 2, which comprises a mixture of at least one fatty acid and polyethylene glycol ester and at least one alkylC8-C30(poly)glycoside.

18. The composition according to claim 3, which comprises a mixture of at least one fatty acid and polyethylene glycol ester and at least one alkylC8-C30(poly)glycoside.

19. The composition according to claim 4, which comprises a mixture of at least one fatty acid and polyethylene glycol ester and at least one alkylC8-C30(poly)glycoside.

20. The composition according to claim 5, which comprises a mixture of at least one fatty acid and polyethylene glycol ester and at least one alkylC8-C30(poly)glycoside.
